(19) United States
(12) Patent Application Publication
(10) Pub. No.: US 2021/0038443 A1
(43) Pub. Date: Feb. 11, 2021

(54) Protective Shield Blocker

(71) Applicant: Alesha Puckett, Lithonia, GA (US)

(72) Inventor: Alesha Puckett, Lithonia, GA (US)

(21) Appl. No.: 17/082,248

(22) Filed: Oct. 28, 2020

Related U.S. Application Data
(63) Continuation-in-part of application No. 15/803,003, filed on Nov. 3, 2017.

(51) Int. Cl. A61F 13/475; A61F 13/53

(52) U.S. Cl. CPC A61F 13/475; A61F 2013/53908; A61F 2013/530481; A61F 13/53

(57) ABSTRACT

The present invention is a disposable feminine hygiene product specially designed to contain menstrual flow leakage in the event a person is encountering heavy or irregular menstrual flow. The Protective Shield Blocker ensures that irregular menstrual flow will be concealed. In an alternative embodiment, the present invention may also be sold in a separate configuration that is ideal for other brands of sanitary products.

### CROSS REFERENCE TO RELATED APPLICATION

This U.S. non-provisional utility application is a continuation in part of U.S. non-provisional application Ser. No. 15/803,003 “Protective Shield Blocker” filed 3 Nov. 2017. The entire contents of f U.S. non-provisional application Ser. No. 15/803,003 “Protective Shield Blocker” filed 3 Nov. 2017 are hereby incorporated into this document by reference.

### FIELD OF INVENTION

The subject disclosure relates to an article for absorbing body exudate. More particularly, the present disclosure relates to a feminine hygiene product for preventing leakage due to irregular menstrual flow by using a specially designed embodiment.

### COPYRIGHT AND TRADEMARK NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever. Trademarks are the property of their respective owners.

### BACKGROUND

The uterus is normally in a straight vertical position. A tipped uterus is a condition where the uterus is tilted backwards toward the back of the pelvis. Women with a retroverted uterus during the menstrual cycle tend to bleed both frontwards and backwards. This condition affects mom than 30% of women worldwide. The cause in most cases are genetic and is entirely normal. Other factors that may contribute to this condition include Pregnancy, Menopause, and Reproductive Problems. Women who have this condition tend to bleed in an irregular direction which then causes soiling of clothing. With the present invention, it will be a process of elimination because the invention will act as a catalyst between a sanitary product and the undergarment. Traditionally, women have relied on products such as disposable sanitary napkins to help control menstrual flow. Disposable sanitary napkins have been inconvenient and unhygienic for individuals who encounter heavy menstrual flow or females who were born with a retroverted or tilted uterus that causes their menstrual cycle to flow in an abnormal direction that leaves conventional sanitary napkins ineffective. This ordeal may result in a person feeling uncomfortable while performing such activities as exercising, working, or even sleeping as an example.

Users who live with this condition whether caused by genetics or other unrelated health concerns are forced to deal with the inconvenience of conventional sanitary napkins. Often, users who have heavy menstrual cycles or irregular menstrual flow have to place tissue or similar material adjacent to the sanitary napkin to prevent leakage which may be time consuming and embarrassing. Although the use of such alternative means is necessary, it may be inconvenient for individuals who use conventional feminine sanitary napkins.

Prior art solutions to similar problems have been focused on using attachment straps such as belts that attach to the user's skin in order to collect fluids. These solutions were not ideal for the user due to the un-comfort caused by form and fit of the pad surface to a user's body as they move.

### BRIEF SUMMARY

The proposed invention is a disposable feminine hygiene product that achieves a dynamic body fit which allows users to benefit from a broader range of protection to combat leakage due to menstrual cycles. More specifically, in accord with one aspect of the Protective Shield Blocker, the proposed hygiene product includes an absorbent pad surface to block menstrual back flow by conveniently placing the Protective Shield Blocker between the user's buttocks for maximum coverage which is where menstrual backflow usually occurs. When the user experiences irregular or heavy menstrual flow, the conventional means of preventing back flow by using sanitary napkins is not affective at protecting against irregular leakage which often occur along the rear portion of sanitary napkins due to limited coverage. The Protective Shield Blocker is positioned specifically in the perineal area to quickly absorb back flowing leakage; this will protect undergarments and outer clothing in the event of such irregular menstrual flow. The key to this novel invention is that it covers areas of a person's body that have not been previously addressed due to a retroverted uterus. More specifically, the current embodiment provides a pad surface and a cord made of an elastic material that protects undergarments and outer clothing. Where menstrual flow has leaked, the Protective Shield Blocker will form a barrier by utilizing the absorbent pad surface that acts as an additional barrier to existing sanitary products. The Protective Shield Blocker will change the way that feminine products are currently used among young girls and women who require special attention as a result of irregular menstrual flow or heavy bleeding.

### BRIEF DESCRIPTION OF THE DRAWINGS

Certain illustrative embodiments and advantages may be best understood by reference detailed description that follows taken in conjunction with the accompanying drawings in which:

FIG. 1 is a diagram of a side-view consistent with certain embodiments of the present invention. The schematic illustrates a fully assembled Protective Shield Blocker, that demonstrates how the pad surface connects to the cord.

FIG. 2 is a diagram of a front-view of the proposed invention consistent with certain embodiments of the present invention.

FIG. 3 is a diagram of a cross-sectional view consistent with certain embodiments of the pad surface of the Protective Shield Blocker.

FIG. 4 is a diagram of a bottom view consistent with certain embodiments of the pad surface of the protective shield blocker.

FIG. 5 is a schematic of a user's perineal area while the Protective Shield Blocker is secured between the wearer's buttocks.

### DETAILED DESCRIPTION OF CERTAIN EMBODIMENTS

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one, or more than one. The term “plurality”, as used herein, is defined as two, or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an exemplary embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

Reference throughout this document to “space” or similar terms means a particular area that make up a structure and is described in connection with the position or the embodiment that is included in an area between two or more components of the present invention. Thus, the appearances of such phrases or in various places throughout this specification are not necessarily all referring to the same embodiment. Furthermore, “space” may be described in any suitable manner by one or more embodiments without limitation.

Reference throughout this document to “arresting” or similar terms indicate a means of stopping leakage from escaping a certain section of the present invention. Thus, the appearances of such phrase or in various places throughout this specification are not necessarily all referring to the same embodiment. Furthermore, “arresting” may be described in any suitable manner by one or more embodiments without limitation.

Reference throughout this document to Protective Shield Blocker or similar terms refer to a feminine hygiene product which serves as an intergluteal extension for absorbing excess menstrual flow. Thus, the appearance of similar phrases throughout this specification may not be limited to just the term Protective Shield Blocker but when the description refers to a means of absorbing menstrual flow then it may be inferred that the description refers to the Protective Shield Blocker.

Reference throughout this document to “panel” refers to the portions of the Protective Shield Blocker that make up the inner and outer wall of the product. Furthermore, the particular features, structures, or characteristics may be combined in any suitable manner in one or more embodiments without limitations.

Reference throughout this document to attachment surface refers to any method of securing the feminine hygiene product to an additional brand of sanitary products such as a sanitary napkin but is not limited to this example. Therefore, where ever this specification refers to any means of attachment it will not be limited to any specific means of attachment but rather any number of attachment methods may be combined to create an attachment for purposes of the present embodiment.

Reference throughout this document to “elastic cord”, “cord” or similar terms refers to an attachment for connecting the pad of the present invention to any number of existing products intended for absorbing human exudate. Therefore, wherever this specification refers to a cord it will not be limited to any specific means of attachment, material or structure but rather any number of attachment methods, materials or structures may be combined to create an attachment for purposes of the present embodiment. In a non-limiting example, the absorbent pad may be sold separately and attached to other sanitary products for both medical as well as non-medical purposes.

Reference throughout this document to “existing sanitary products” refers to any number of traditional sanitary products that are commonly used during menstrual cycles. Therefore, wherever this specification refers to existing sanitary products it will not be limited to any specific product but rather any number of sanitary products and structures may be combined for purposes of the present embodiment. In a non-limiting example, the existing sanitary product may be in reference to a sanitary napkin or tampon but is not limited to these examples.

Reference throughout this document to “landing” refers to a structure that is used to attach the cord to an additional sanitary product. Therefore, wherever this specification refers to a landing it will not be limited to any specific platform, size or shape but rather any number of platforms, sizes and shapes may be combined for purposes of the present embodiment. In a non-limiting example, the landing may be a flat rectangular platform but is not limited to this example.

The material that a feminine product is made of is an essential aspect of the appealability of the product because it may be a predictability of a user's experience when wearing the product while sleeping, exercising, or just sitting. From a convenience perspective, the aspects of the product's comfort which affects the user's sensory factors are an important attribute for effectively presenting a more inviting experience during an otherwise uncomfortable time of the month. In a non-limiting example, because odors emit from surfaces of such products then a combination of scents and special liners may be used to deal with odors and release scents that are pleasant to the user. Furthermore, in an additional non-limiting example, the liner of the absorbent pad surface may be made to release pleasant hypoallergenic fragrances to help eliminate odors.

The product is utilized by applying the extension in the natal cleft area of the wearer to help absorb menstrual fluids for those who often suffer from heavy menstrual cycles, fibroids, or an introverted uterus that causes the menstrual discharge to flow backwards.

In the exemplary embodiment, the current invention may consist of a method of attaching the cord structure to other surfaces for an alternate use. The method may encompass any number of commonly used methods in which similar products have been attached. For the sake of the current invention the method of attachment will not be limited to a certain method.

In an additional embodiment, the Protective Shield Blocker may be marketed as a different configuration in that the absorbent pad portion may be separately sold in various shapes and sizes to consumers such as maxi, overnight, and mini. The Protective Shield Blocker uses a soft dry weave material that fully fits along the curves of a user's perineal area by contouring to a user's skin to create a seal which prevents menstrual flow from escaping as a result of back flow. The absorbent pad surface allows the user to move freely without feeling uncomfortable. In an alternate embodiment, the Protective Shield Blocker may also be utilized for a variety of medical purposes for both humans as well as veterinarian purposes. As a non-limiting example, a version of the Protective Shield Blocker may be used on male patients during post-surgery for such ailments as prostate cancer or other medical purposes such as absorbing human exudate from male's penis or generally in the male genitalia area.

In a non-limiting example, the pad surface may include absorbent core material, and a scented hypoallergenic component that may be evenly distributed to act as a releasing agent for a more pleasant scent. The pad component may also include a water proof laminated outer surface that in a non-limiting example may be made out of non-woven material which incorporates an appealing design. This outer surface helps to prevent any fluids from leaving the pad mainly through the surface of the external surface which could be a significant inconvenience for the user. This is important to allow a main barrier of protection in an effort to contain menstrual flow inside the absorbent core of the Protective Shield Blocker as it exits the body. The optional cord is an important aspect of the configuration because it acts as means of attaching the pad component to existing sanitary products for increased options for consumers.

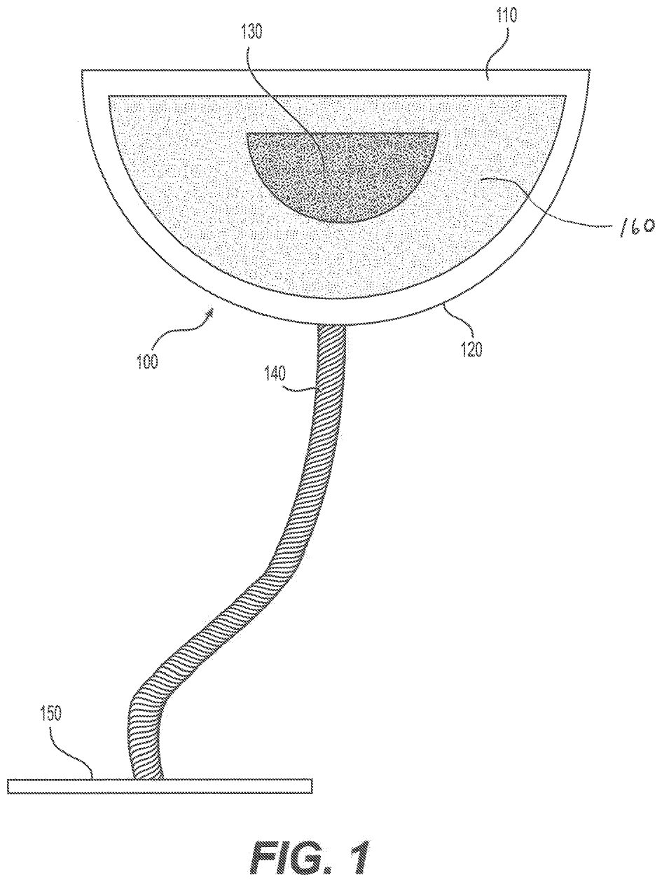

Turning now to FIG. 1, this figure presents a side-view of the Protective Shield Blocker 100 for an exemplary illustration to include the elastic cord 140. The Protective Shield Blocker 100 may be securely attached to other existing sanitary products by attaching the cord 140 by use of the landing 150. The Protective Shield Blocker has an upper permeable section 110 and a lower impermeable section 120. The schematic further presents an absorbent material 130 to ensure that menstrual flow is adequately contained. The transfer layer 160 is designed to allow the fluids to freely flow toward the absorbent material 130. The wearer must attach the landing 150 to an existing sanitary product if necessary. The figure depicts the side-view of a fully assembled Protective Shield Blocker 100.

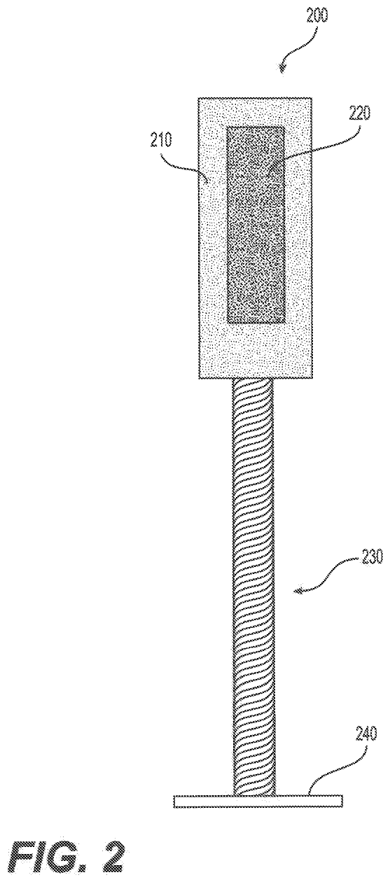

Turning now to FIG. 2, this figure presents a front-view 200 of the pad section of the protective shield blocker for an exemplary view of the absorbent pad material 220 and the surrounding pad surface 210. The absorbent pad material 220 serves as the focal point for menstrual containment if the menstrual flow has breached an existing sanitary product such as a sanitary napkin. The remaining pad surface 210 acts as additional absorbent material to ensure that menstrual flow is adequately contained. The elastic cord 230 serves to connect the landing surface to the pad surface. The landing 240 serves as the attachment surface between an existing sanitary product and the Protective Shield Blocker.

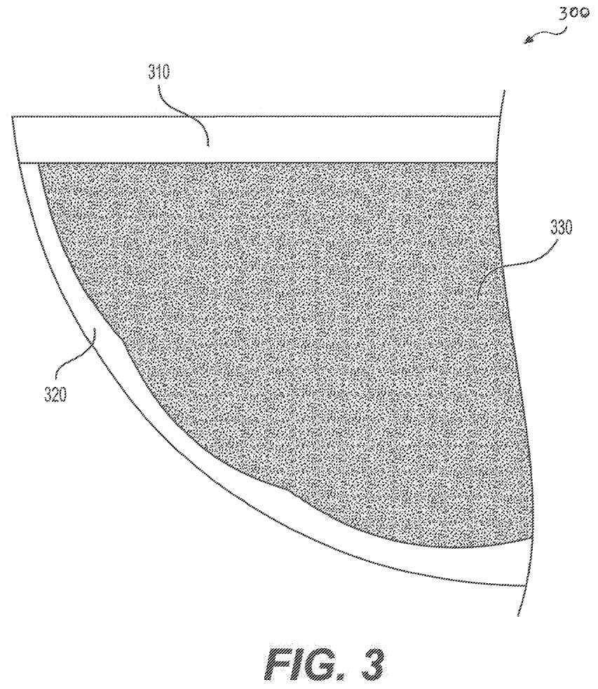

Turning now to FIG. 3, this figure presents a cross-sectional view of the Protective Shield Blocker 300 for an exemplary illustration of the upper permeable section 310 and the lower impermeable section 320. The schematic further presents an absorbent core material 330. The upper permeable section 310 allows fluids to pass through the upper material where it is absorbed by the core material 330. As the bodily fluids are absorbed the fluids are eventually arrested against the lower impermeable section 320 which acts as a barrier and does not allow fluids to pass beyond the outer surface of the Protective Shield Blocker.

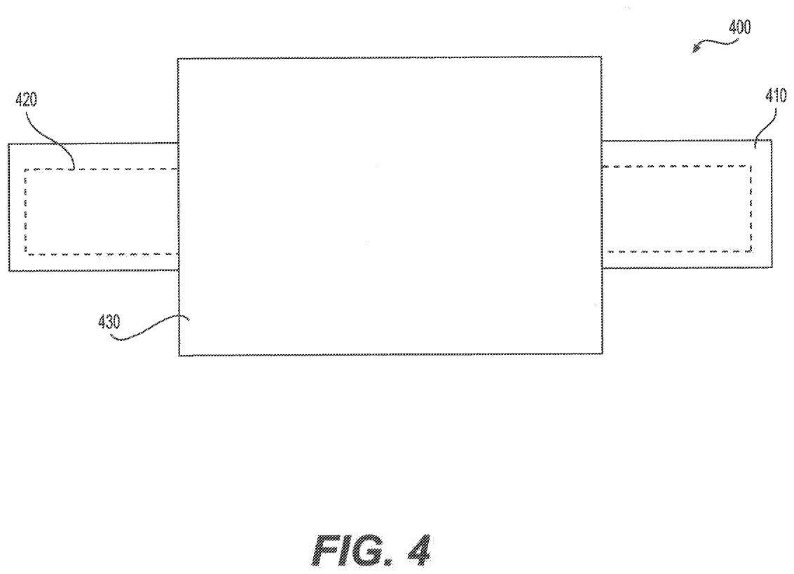

Turning now to FIG. 4, this figure presents a bottom view of the Protective Shield Blocker 400 for an exemplary illustration of the Protective Shield Blocker depicting the under body of the absorbent pad to include the absorbent material 420, transfer layer 410, and landing 430. The schematic further presents a bottom view of the connecting surface of the landing 430. The landing 430 may be placed on the mating surface of existing sanitary products. The landing 430 forms a bond with the existing sanitary product.

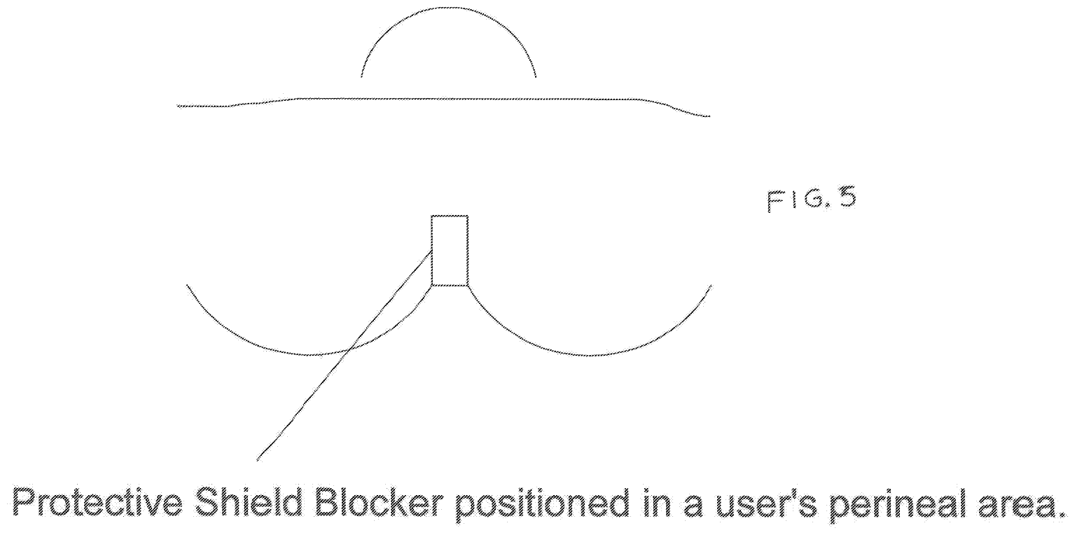

Turning now to FIG. 5, this figure presents a view of a user's perineal area while the Protective Shield Blocker is secured between the wearer's buttocks. The schematic depicts what an embodiment of the Protective Shield Blocker would look like if positioned without the presence of the elastic cord. This depiction is merely intended to provide a better understanding of where the Protective Shield Blocker will be placed when in use.

The embodiment includes a resin bonded non-woven member that is positioned adjacent to the absorbent material which acts as a conduit for fluid. Such carded nonwoven members have a more uniform fiber alignment than other traditional nonwoven products which result in greater stability and internal bond strength. Resin bonding of carded nonwoven fabric improves its wetted tensile strength and also improves the flow of fluids in comparison to traditional fiber membranes. The carded nonwoven resin constitutes the transfer layer which is continuous with the absorbent core.

In this specification the term “continuous with” is defined to mean in contact with so that if [A] and [B] are susceptible to an event or treatment, then “[A] is continuous with [B]” shall mean that an event or treatment applied to [A] will eventually act at least partially upon [B] and vice versa, and so that if [A] and [B] are both absorbent materials, then a fluid absorbed by [A] will eventually be diffused into at least a portion of [B] and vice versa.

In the instant embodiment it is important to note that the embodiment may be designed to either be attached to an existing sanitary product such as a tampon or menstrual cups. In the case where a user decides to purchase an embodiment of a Protective Shield Blocker that can be attached to an existing sanitary product, the landing attachment will include a surface that may be attached mechanically or chemically to the exterior of the existing sanitary product in a way that allows the landing to be applied using structures and adhesives that are gentle on a user's skin. For instance, the landing surface may include an adhesive tape such as a hypoallergenic strip for attaching to the existing product without the user experiencing allergic reactions or other irritations. Alternately, other kinds of fasteners attachments, or combinations may be used, including clips, hook-and-loop fabric, snaps, or fabric extensions such as wings which may be folded around to incorporate a portion of the product. As an example, if a feminine napkin is being used during menses, a user may decide to couple or attach the intergluteal extension to the feminine napkin by applying the landing to the napkin in a suitable location by attaching the bottom surface of the landing attachment to an appropriate surface of the feminine napkin. As another alternative, if the user finds it undesirable to couple an intergluteal extension to an existing sanitary product, then the user may employ a version of the embodiment which does not include such an attachment option and which will not include the cord nor the landing attachment. This version is useful for users who use products such as tampons or desire only to use the intergluteal extension alone, without attaching it to any other existing sanitary product.

In an exemplary embodiment, the cord may be selected from materials such as absorbent gel material which expands and effectively absorbs fluids when it is in contact with menstrual exudate. Once the embodiment is placed along the gluteal area of a person's body the cord will retain its original size until the cord is in contact with liquids and thereafter may expand as it absorbs bodily fluids. The combination of the absorbent cord and the soft body will greatly assist the embodiment in containing the menstrual backflow, because the cord is advantageously located in an area along the perineum which is between the vagina and the rectum. This is ideal because absorbency of the cord acting in conjunction with the soft body may act as a dual absorption system. Additionally, since women of various sizes will be wearing the Protective Shield Blocker then it is important note that the expansion of the cord will put it in contact with areas that would not otherwise be covered by traditional product, depending on a person's size and shape.

According to an alternative embodiment within the scope of the invention, the landing attachment further comprises a surface attachable to a sanitary product such as a knot in the cord, or a dip, or a hook. Alternatively, the landing attachment may also further comprises a temporary adhesive such as those containing volatile hydrocarbons compounded with heptane, whether as a linear, branched, or cyclic isomer, and methyl acetate. The volatile compounds evaporate overtime, leaving a tacky or sticky material which may effectively form a temporary bond between fabric materials such as clothing.

While certain features and aspects have been described with respect to exemplary embodiments, one skilled in the art will recognize that numerous modifications are possible. Further, while various methods and processes described herein may be described with respect to particular structural and/or functional components for ease of description, methods provided by various embodiments are not limited to any particular structural and/or functional architecture. Unauthorized instances of apparatuses and methods claimed herein are to be considered infringing, no matter where in the world they are advertised, sold, offered for sale, used, possessed, or performed.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled.

Hence, while various embodiments are described with or without certain features for ease of description and to illustrate exemplary aspects of those embodiments, the various components and/or features described herein with respect to a particular embodiment may be substituted, added, and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A intergluteal extension for absorbing menstrual back flow, comprising:
a body comprising
a permeable upper panel having a first surface facing toward a user's body, said permeable upper panel continuous with a first transfer layer,
an impermeable lower panel continuous with said transfer layer, said lower panel opposing said upper panel and having a second surface facing away from the user's body when in use,
an absorbent core material deposited between said upper panel and said lower panel of said body,
a landing attachment, and
a cord having a first end connected to and extending from said lower panel and a second end connected to said landing attachment.

2. The intergluteal extension of claim 1, wherein said external surface of said upper panel further comprises a resin bonded fiber membrane continuous with said transfer layer.

3. The intergluteal extension of claim 2, wherein said resin bonded fiber membrane further comprises a second transfer layer, and said permeable upper panel is continuous with said second transfer layer.

4. The intergluteal extension according of claim 1, wherein the entirety of said external surface of said upper panel is continuous with said lower panel.

5. The intergluteal extension of claim 1, wherein said absorbent core is continuous with said transfer layer.

6. The intergluteal extension of claim 5, wherein said absorbent core retains a first portion of fluid absorbed from said upper layer and passes a second portion of fluid absorbed from said upper layer into said lower layer.

10. The intergluteal extension of claim 1, wherein said cord further comprises an elastic material.

11. The intergluteal extension of claim 1, wherein said cord comprises a material that expands upon absorbing a fluid.

12. The intergluteal extension of claim 1, wherein said elastic cord further comprises a dry granular material which becomes an absorbent gel upon contact with a fluid.

13. The intergluteal extension of claim 1, wherein said landing attachment further comprises a surface attachable to a sanitary product.

14. The intergluteal extension of claim 13, wherein said landing attachment further comprises a temporary adhesive.

15. The intergluteal extension of claim 14, wherein said temporary adhesive comprises a heptane.
